(19) United States
(12) Patent Application Publication
(10) Pub. No.: US 2026/0209253 A1
(43) Pub. Date: Jul. 23, 2026

(54) SILVER ION COMPLEX GEMINI QUATERNARY AMMONIUM SALT: PREPARATION METHOD AND APPLICATIONS

(71) Applicant: ANHUI KIWI BIOTECH CO., LTD, Anhui (CN)

(72) Inventors: Hongtao KONG, Anhui (CN); Hui MIAO, Anhui (CN); Xiangchuan XU, Anhui (CN); Zhe LIU, Anhui (CN); Ruixia WANG, Anhui (CN)

(73) Assignee: ANHUI KIWI BIOTECH CO., LTD, Anhui (CN)

(21) Appl. No.: 18/716,513

(22) PCT Filed: Jan. 24, 2024
(86) PCT No.: PCT/CN2024/073778

§ 371 Date: Jun. 5, 2024

(30) Foreign Application Priority Data
Apr. 11, 2023 (CN) 202310378671.0

(51) Int. Cl. C07F 1/00; A01N 55/02; A01P 1/00

(52) U.S. Cl. CPC C07F 1/005; A01N 55/02; A01P 1/00

(57) ABSTRACT

The present invention provides a silver ion complex Gemini quaternary ammonium salt along with its preparation method and application. The preparation method includes: the Michael addition reaction and nucleophilic substitution reaction are conducted on dimethylaminoethyl acrylate, long chain thiol and dihaloalkane to obtain Gemini quaternary ammonium salt; and then the Gemini quaternary ammonium salt combines with silver salt to yield the silver ion complexed Gemini quaternary ammonium salt. The present invention includes the silver ion complex of Gemini quaternary ammonium salt and the preparation method and the application. The silver ion complex of Gemini quaternary ammonium salt has higher sterilization and environmental degradability, which greatly reduces the probability of bacterial resistance.

### CROSS-REFERENCE TO RELATED APPLICATIONS

This application claims priority to Chinese Patent Application No. 202310378671.0 entitled “Silver Ion Complex Gemini Quaternary Ammonium Salt: Preparation Method and Applications,” filed on Apr. 11, 2023. The entirety of the aforementioned application is incorporated herein by reference for all purposes.

### TECHNICAL FIELD

The present disclosure relates to the field of sterilization and disinfectants. More specifically, it pertains to a novel silver ion complex Gemini quaternary ammonium salt and discusses both the method for its preparation and its various applications.

### DESCRIPTION OF RELATED ART

Quaternary ammonium compounds (QACs), recognized as effective sterilizing agents, have been extensively utilized in diverse fields including industry, agriculture, construction, food services, textiles, and healthcare. Despite their widespread use and stable chemical structure, QACs have been increasingly found to accumulate in the environment and water bodies. This accumulation has become more significant following the COVID-19 pandemic, with a marked increase in QAC usage leading to about 90% of environmental dust now containing these compounds, approximately twice the amount observed pre-pandemic.

The persistence of QACs in the environment can induce resistance in bacteria over time. Additionally, at higher concentrations, QACs may serve as a nutrient source, fostering rapid bacterial growth. The emergence of resistant bacteria poses a substantial threat to human health, necessitating the urgent development of new sterilizing agents. These new agents should not only be more effective at lower concentrations but also capable of degradation to reduce the risk of promoting antibiotic resistance.

Gemini quaternary ammonium salts, characterized by their unique dual hydrophilic quaternary ammonium functional groups, demonstrate the ability to adsorb more swiftly onto bacterial surfaces, thereby enhancing sterilization efficiency. This feature makes them particularly significant in the development of disinfectants that are not only highly effective but also biodegradable. Such products are likely to find broad application and hold substantial commercial value, especially as environmental safety becomes a paramount concern.

### SUMMARY

To solve the above technical issues, the present invention provides a silver ion complex Gemini quaternary ammonium salt along with its preparation and application. The silver ion complex Gemini quaternary ammonium salt presented herein exhibits highly efficient sterilizing performance and can rapidly degrade in the environment, thereby reducing the chance of bacterial resistance development.

The silver ion complex Gemini quaternary ammonium salt proposed by the present invention is structured as follows:

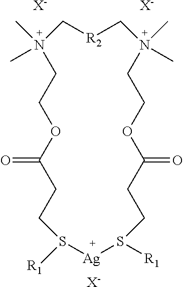

R₁, which is a saturated straight-chain alkyl group having 6-12 carbon atoms; R₂, which is an alkyl group having 1-8 carbon atoms; X, which is an anionic group.

Preferably, R₁ is saturated straight chain alkyl group having 8 carbon atoms; Preferably, R₂ is methylene group.

Preferably, X is NO₃, OAc, SCN, BF₄, OTf, HSO₄, PF₆, BPh₄, Cl, Br, or I;

The present invention also proposes a method of preparation of silver ion complex Gemini quaternary ammonium salt, with the following steps:

Step 1. Performing a Michael addition reaction and a nucleophilic substitution reaction on dimethylaminoethyl acrylate, a long-chain thiol, and a dihaloalkane to obtain the Gemini quaternary ammonium salt;

Step 2. Reacting the obtained Gemini quaternary ammonium salt with a silver salt to form the silver ion complexed Gemini quaternary ammonium salt.

Preferably, the structural formula of the long-chain thiol is:

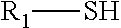

In which, R₁ is a saturated straight-chain alkyl group having 6-12 carbon atoms;
Preferably, the long-chain thiol is 1-Hexanethiol, 1-Heptanthiol, 1-Octanethiol, 1-Nononythiol, or 1-Decanethiol;
Preferably, the structure formula of the dihaloalkane is:

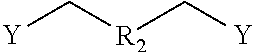

In which, R₂ is an alkyl group having 1-8 carbon atoms, and Y is Cl, Br, or I;

Preferably, the dihaloalkane is 1, 3-dichloropropane or 1, 3-dibromopropane.

The synthesis of the silver ion complex Gemini quaternary ammonium salt is as follows:

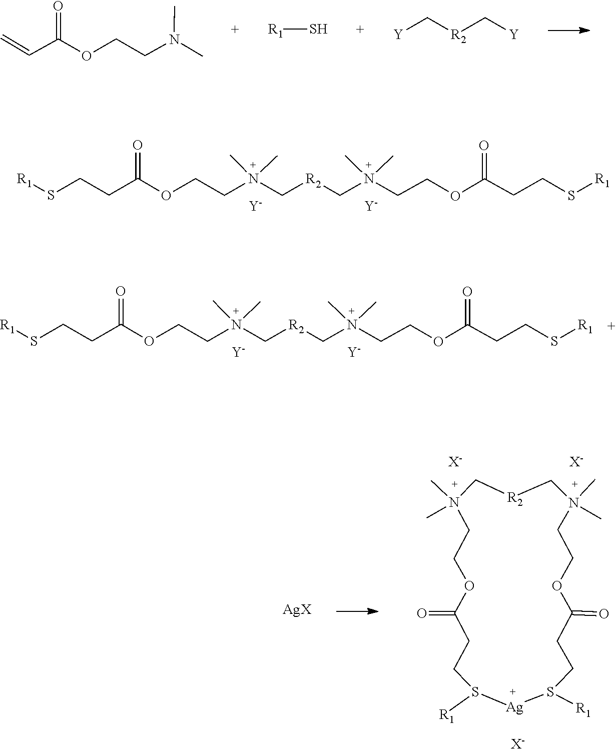

Preferably, in step 1, the reaction temperature is 50-150° C. and the reaction time is between 6-18 h;

Preferably, in step 2, the silver salt is silver nitrate;

Preferably, the molar ratio of silver salt to Gemini quaternary ammonium salt is at least

3:1

Preferably, in step 2, the reaction temperature is 10-30° C. and the reaction time is between 6-18 h.

The present invention also proposes the application of the silver ion complex Gemini quaternary ammonium salt prepared by the aforementioned method as a sterilizing agent.

The technical advantages of the present invention:
1. The silver ion complex Gemini quaternary ammonium salt described in this application has two quaternary ammonium functional groups in its structure, which can more easily adsorb onto bacterial surfaces, thereby possessing higher bactericidal activity. Meanwhile the ester groups in the molecular structure described in this application can be hydrolyzed under natural conditions, thereby avoiding increase of bacterial resistance resulting from prolonged exposure to the QACs environment.
2. The silver ion complex Gemini quaternary ammonium salt described in this application can further enhance sterilization efficiency through its unique thioether bond and silver ion complexation. On one hand, this complexation structure transforms the quaternary ammonium salt into a rigid structure. The greater the molecular rigidity of the quaternary ammonium salt, the easier it is for the hydrophobic ends to insert into bacteria, thus leading to more thorough disruption of bacterial cell membranes. On the other hand, after the silver ion complex Gemini quaternary ammonium salt passes through the cell membrane, Ag⁺ can readily form complexes with intracellular —SH and —NH₂ groups (such as those on intracellular enzymes and the nitrogenous bases of DNA/RNA), thereby inhibiting and disrupting normal bacterial metabolic activities and further killing bacteria more effectively. Moreover, with the improved sterilization efficiency of the silver ion complex Gemini quaternary ammonium salt, it becomes possible to reduce the concentration of quaternary ammonium salt used during practical application, consequently lowering the amount released into the natural environment and further reducing the probability of bacteria resistance.
3. The preparation method of silver ion complex Gemini quaternary ammonium salt described in this application has the advantages of cheap raw materials and easy availability, simple synthesis process, mild reaction conditions, easy post-treatment, and high yield.

### BRIEF DESCRIPTION OF THE DRAWINGS

FIG. 1 shows the 1H NMR spectra of the silver ion complex Gemini quaternary ammonium salt obtained from the reaction between C6 chloro Gemini quaternary ammonium salts and AgNO₃ at different molar ratios.

FIG. 2 shows the 1H NMR spectrum of C8 chloro Gemini quaternary ammonium salt.

FIG. 3 shows the 1H NMR spectra of non-complexed silver ion Gemini quaternary ammonium salts obtained by C10 chloro Gemini quaternary ammonium salts without sulfur and AgNO₃ at different molar ratios.

### DESCRIPTION OF THE EMBODIMENTS

The application provides a detailed explanation of the disclosed technical solutions through specific embodiments as below. However, it should be explicitly stated that these embodiments are presented for illustrative purposes and should not be construed as limiting the scope of this application.

### Example 1

This example proposes a silver ion complex Gemini quaternary ammonium salt, with the following structural formula:

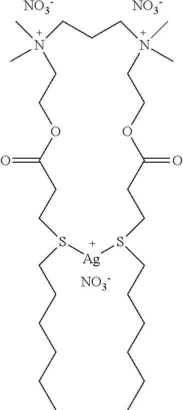

The above silver ion complex Gemini quaternary ammonium salt was prepared as the following steps:
(1) According to the molar ratio of 2:2:1, dimethylaminoethyl acrylate, 1-hexyl mercaptan, and 1, 3-dichloropropane were added into the pressure bottle, and then isopropyl alcohol was added as solvent. After stirring reaction at 120° C. for 12 h, the solvent was removed by vacuum distillation. The resulting product was washed with acetone for three times and freeze-dried to obtain the Gemini quaternary ammonium salt. The Gemini quaternary ammonium salt was C6 chloro Gemini quaternary ammonium salt, which was denoted by the code C6-S—Cl. The structural formula of the C6 chloro Gemini quaternary ammonium salt is as follows:

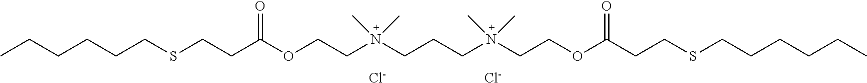

(2) According to the molar ratio of 1:3, the above C6 chloro Gemini quaternary ammonium salt and AgNO₃ were added to water to form an aqueous solution with a concentration of 1.0 wt %. After stirring for 12 h, the generated AgCl was filtered and removed to obtain the supernatant. Thus the aqueous solution of the silver ion complex Gemini quaternary ammonium salt was obtained, which was denoted by the code C6-S—NO₃—Ag⁺.

In order to verify whether there was a complex reaction between Ag⁺ and the C6-S—Cl, the C6-S—Cl and AgNO₃ were added into the water according to the molar ratio of 1:1 and 1:2 respectively, and the operation was performed according to step (2). The complexes of C6-S—Cl with molar ratio of 1:1 and 1:2 to the AgNO₃ were obtained, respectively.

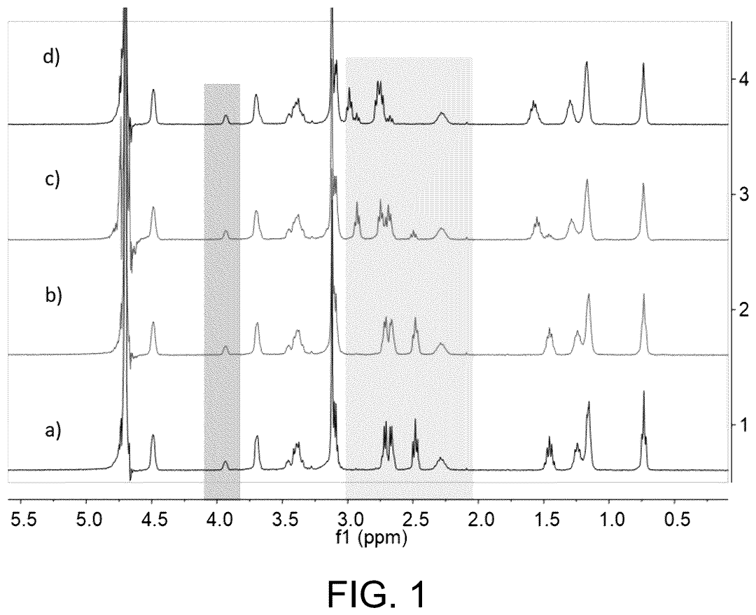

The silver ion complexes Gemini quaternary ammonium salt obtained from the reaction of C6-S—Cl with molar ratio of 1:0, 1:1, 1:2 and 1:3 with AgNO₃ was tested by 1H NMR spectrum. The results were shown in FIG. 1. The silver ion complexes Gemini quaternary ammonium salt obtained from the reaction of C6-S—Cl with molar ratio of 1:0 and 1:3 are C6-S—Cl and C6-S—NO₃—Ag⁺.

FIG. 1 showed the 1H NMR spectra of C6-S—Cl with different molar ratios obtained by reacting with AgNO₃. Among them, a) was the spectra of C6-S—Cl with molar ratio of 1:0 and AgNO₃, actually was the spectra of C6-S—Cl; b) was the spectra of C6-S—Cl with molar ratio 1:1 with AgNO₃; c) was the spectra of C6-S—Cl with molar ratio 1:2 with AgNO₃; d) was the spectra of C6-S—Cl with molar ratio 1:3 with AgNO₃, which is C6-S—NO₃—Ag⁺.

From FIG. 1, it could be observed that during the reaction between C6-S—Cl and AgNO₃ to form the silver ion complex Gemini quaternary ammonium salt, initially added AgNO₃ mainly reacted with the chloride ions in the C6-S—Cl to form AgCl. Comparing the 1H NMR spectra before and after the addition of AgNO₃, it was noted that there was no significant change, indicating that Ag⁺ and the C6-S—Cl had not yet undergone complexation reaction. However, as the amount of AgNO₃ increases, in addition to reacting with the chloride ions in the C6-S—Cl to form AgCl, some Ag⁺ ions began to undergo complexation with C6-S—Cl. Comparing the 1H NMR spectra before and after the addition of AgNO₃, it was observed that there was a significant left shift in the peak around 2.7, corresponding to the hydrogen atoms of the methylene group connected to S in the C6-S—Cl. This indicated that complexation reaction had occurred between Ag⁺ and C6-S—Cl.

### Example 2

This example proposed a silver ion complex Gemini quaternary ammonium salt, with the following structural formula:

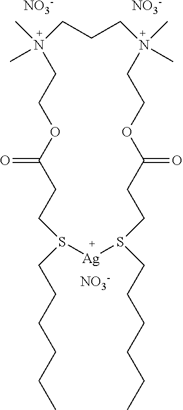

The above silver ion complex Gemini quaternary ammonium salt was prepared as the following steps:
(1) According to the molar ratio of 2:2:1, Dimethylaminoethyl acrylate, 1-hexyl mercaptan and 1, 3-dibromopropane were added to the pressure bottle and isopropyl alcohol was added as the solvent. After stirring at 50° C. for 12 h, the solvent was removed by vacuum distillation, and the resulting product was washed with acetone three times and freeze-dried to obtain Gemini quaternary ammonium salt. The Gemini quaternary ammonium salt was C6 Bromo Gemini quaternary ammonium salt, denoted by the code C6-S—Br. The structural formula of the C6 bromine quaternary ammonium salt was as follows:

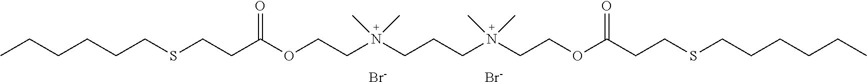

(2) According to the molar ratio of 1:3, the above C6 bromo Gemini quaternary ammonium salt and AgNO₃ were added into water to form an aqueous solution with a concentration of 1.0 wt %. After stirring for 12 h, the generated AgBr was filtered and removed to obtain the supernatant. Thus the aqueous solution of the silver ion complex Gemini quaternary ammonium salt was obtained, which was denoted by the code C6-S—NO₃—Ag⁺.

### Example 3

This example proposed a silver ion complex Gemini quaternary ammonium salt, with the following structural formula:

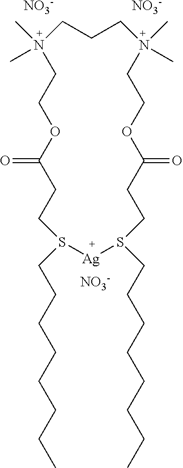

The above silver ion complex Gemini quaternary ammonium salt was prepared as the following steps:
(1) According to the molar ratio of 2:2:1, dimethylaminoethyl acrylate, 1-octylmercaptan, and 1, 3-dichloropropane were added into the pressure bottle, and then isopropyl alcohol was added as solvent. After stirring reaction at 120° C. for 12 h, the solvent was removed by vacuum distillation. The resulting product was washed with acetone for three times and freeze-dried to obtain the Gemini quaternary ammonium salt. The Gemini quaternary ammonium salt was C8 chloro Gemini quaternary ammonium salt, which was denoted by the code C8-S—Cl. The structural formula of the C8 chloro Gemini quaternary ammonium salt is as follows:

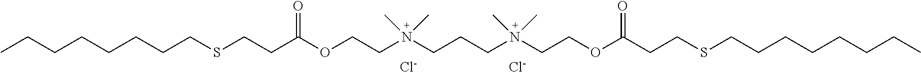

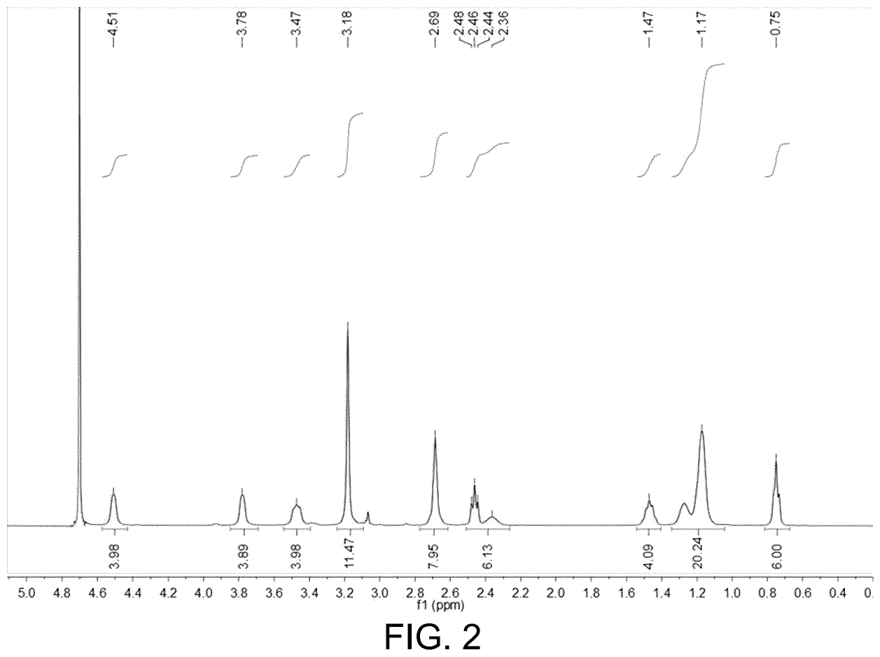

The 1H NMR spectrum of the C8 chloro Gemini quaternary ammonium salt was shown in FIG. 2.
(2) According to the molar ratio of 1:3, the above C8 chloro Gemini quaternary ammonium salt and AgNO₃ were added to water to form an aqueous solution with a concentration of 1.0 wt %. After stirring for 12 h, the generated AgCl was filtered and removed to obtain the supernatant. Thus, the aqueous solution of the silver ion complex Gemini quaternary ammonium salt was obtained, which was denoted by the code C8-S—NO₃—Ag⁺.

### Example 4

This Example proposed a silver ion complex Gemini quaternary ammonium salt, with the following structural formula:

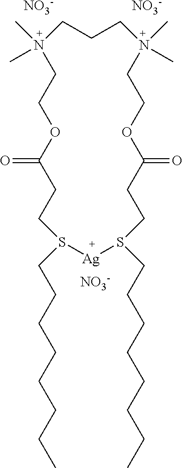

The above silver ion complex Gemini quaternary ammonium salt was prepared as the following steps:
(1) According to the molar ratio of 2:2:1, dimethylaminoethyl acrylate, 1-octylmercaptan, and 1, 3-dibromoropropane were added into the pressure bottle, and then isopropyl alcohol was added as solvent. After stirring reaction at 50° C. for 12 h, the solvent was removed by vacuum distillation. The resulting product was washed with acetone for three times and freeze-dried to obtain the Gemini quaternary ammonium salt. The Gemini quaternary ammonium salt was C8 bromo Gemini quaternary ammonium salt, which was denoted by the code C8-S—Br. The structural formula of the C8 bromo Gemini quaternary ammonium salt was as follows:

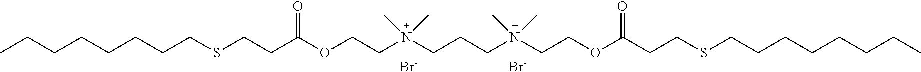

(2) According to the molar ratio of 1:3, the above C8 bromo Gemini quaternary ammonium salt and AgNO₃ were added to water to form an aqueous solution with a concentration of 1.0 wt %. After stirring for 12 h, the generated AgBr was filtered and removed to obtain the supernatant. Thus, the aqueous solution of the silver ion complex Gemini quaternary ammonium salt was obtained, which was denoted by the code C8-S—NO₃—Ag⁺.

### Example 5

This Example proposed a silver ion complex Gemini quaternary ammonium salt, with the following structural formula:

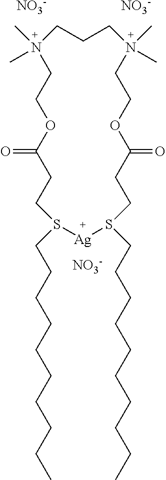

The above silver ion complex Gemini quaternary ammonium salt was prepared as the following steps:
(1) According to the molar ratio of 2:2:1, dimethylaminoethyl acrylate, 1-decyl mercaptan, and 1, 3-dichloropropane were added into the pressure bottle, and then isopropyl alcohol was added as solvent. After stirring reaction at 120° C. for 12 h, the solvent was removed by vacuum distillation. The resulting product was washed with acetone for three times and freeze-dried to obtain the Gemini quaternary ammonium salt. The Gemini quaternary ammonium salt was C10 chloro Gemini quaternary ammonium salt, which was denoted by the code C10-S—Cl.

The structural formula of the C10 chloro quaternary ammonium salt was as follows:

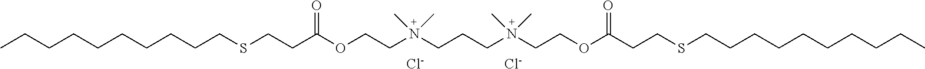

(2) According to the molar ratio of 1:3, the above C10 chloro Gemini quaternary ammonium salt and AgNO₃ were added to water to form an aqueous solution with a concentration of 1.0 wt %. After stirring for 12 h, the generated AgCl was filtered and removed to obtain the supernatant. Thus, the aqueous solution of the silver ion complex Gemini quaternary ammonium salt was obtained, which was denoted by the code C10-S—NO₃—Ag⁺.

### Example 6

This Example presented a silver ion complex Gemini quaternary ammonium salt, with the following structural formula:

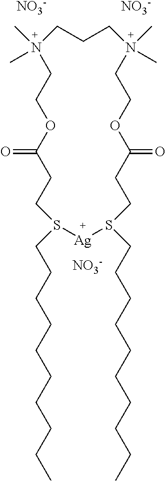

The above silver ion complex Gemini quaternary ammonium salt was prepared as the following steps:
(1) According to the molar ratio of 2:2:1, dimethylaminoethyl acrylate, 1-decyl mercaptan, and 1, 3-dibromoropropane were added into the pressure bottle, and then isopropyl alcohol was added as solvent. After stirring reaction at 50° C. for 12 h, the solvent was removed by vacuum distillation. The resulting product was washed with acetone for three times and freeze-dried to obtain the Gemini quaternary ammonium salt. The Gemini quaternary ammonium salt was C10 bromo Gemini quaternary ammonium salt, which was denoted by the code C10-S—Br. The structural formula of the C10 bromo quaternary ammonium salt was as follows:

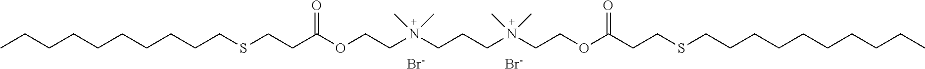

(2) According to the molar ratio of 1:3, the above C10 bromo Gemini quaternary ammonium salt and AgNO₃ were added to water to form an aqueous solution with a concentration of 1.0 wt %. After stirring for 12 h, the generated AgBr was filtered and removed to obtain the supernatant. Thus, the aqueous solution of the silver ion complex Gemini quaternary ammonium salt was obtained, which was denoted by the code C10-S—NO₃—Ag⁺.

### Example 7

The present Example presented a C8 nitrate type Gemini quaternary ammonium salt, with the following structural formula:

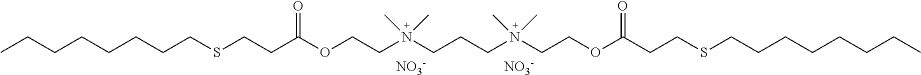

(1) According to the molar ratio of 2:2:1, dimethylaminoethyl acrylate, 1-octylmercaptan, 1, 3-dichloropropane were added into the pressure resistant bottle, and then isopropanol was added as the solvent. After stirring reaction at 120° C. for 12 h, the solvent was removed by vacuum distillation, and the resulting product was washed three times with acetone and freeze-dried to obtain C8 chloro Gemini quaternary ammonium salt.
(2) According to the molar ratio of 1:2, the above C8 chloro Gemini quaternary ammonium salt and AgNO₃ were added to water, prepared into an aqueous solution with a concentration of 1 wt %, stirred for 12 h, filtered to remove the generated AgCl and take the supernatant, that was, the C8 nitrate type Gemini quaternary ammonium salt was obtained, denoted by the code C8-S—NO₃.

### Comparative Example 1

In this paper, a kind of copper ion complexed Gemini quaternary ammonium salt was proposed. The preparation method included the following steps:
(1) According to the molar ratio of 2:2:1, dimethylaminoethyl acrylate, 1-octylmercaptan, and 1, 3-dichloropropane were added into the pressure bottle, and isopropyl alcohol was added as the solvent. After stirring reaction at 120° C. for 12 h, the solvent was removed by vacuum distillation, and the resulting product was washed with acetone three times and freeze-dried to obtain Gemini quaternary ammonium salt. The Gemini quaternary ammonium salt was C8 chloro quaternary ammonium salt.
(2) According to the 1:1 molar ratio, the C8 chloro Gemini quaternary ammonium salt and CuCl₂ were added to water, prepared into an aqueous solution with a concentration of 1.0 wt %, stirred for 12 h, and filtered to take the supernatant, that was, the aqueous solution of the copper ion complex Gemini quaternary ammonium salt was obtained, denoted by the code C8-S—Cl—Cu²⁺.

### Comparative Example 2

In this paper, a manganese ion complex Gemini quaternary ammonium salt was proposed. The preparation method included the following steps:
(1) According to the molar ratio of 2:2:1, dimethylaminoethyl acrylate, 1-octylmercaptan, and 1, 3-dichloropropane were added into the pressure bottle, and isopropyl alcohol was added as the solvent. After stirring reaction at 120° C. for 12 h, the solvent was removed by vacuum distillation, and the resulting product was washed with acetone three times and freeze-dried to obtain Gemini quaternary ammonium salt. The Gemini quaternary ammonium salt was C8 chloro quaternary ammonium salt.
(2) The above C8 chloro quaternary ammonium salt and MnCl₂ were added to water according to the molar ratio of 1:1, prepared into an aqueous solution with a concentration of 1.0 wt %, stirred and reacted for 12 h, and the supernatant was taken after filtration to obtain the aqueous solution of the manganese ion complex Gemini quaternary ammonium salt, denoted by the code C8-S—Cl—Mn²⁺.

### Comparative Example 3

In this paper, a kind of iron-ion complexed Gemini quaternary ammonium salt was proposed. The preparation method includes the following steps:
(1) According to the molar ratio of 2:2:1, dimethylaminoethyl acrylate, 1-octylmercaptan, 1, 3-dichloropropane were added into the pressure bottle, and isopropyl alcohol was added as the solvent. After stirring reaction at 120° C. for 12 h, the solvent was removed by vacuum distillation, and the resulting product was washed with acetone three times and freeze-dried to obtain Gemini quaternary ammonium salt. The Gemini quaternary ammonium salt was C8 chloro quaternary ammonium salt.
(2) The above C8 chloro quaternary ammonium salt and FeCl₃ were added to water according to the molar ratio of 1:1 to form an aqueous solution with a concentration of 1.0 wt %. After stirring and reaction for 12 h, the supernatant was taken after filtration to obtain the aqueous solution of the iron ion complex Gemini quaternary ammonium salt, which was denoted by the code C8-S—Cl—Fe³⁺.

### Comparative Example 4

In this paper, a kind of uncomplexed silver ion Gemini quaternary ammonium salt was proposed. The preparation method included the following steps:
(1) Decanoyl chloride was slowly dropped into the acetonitrile solution of dimethylaminoethanol in the ice water bath according to the molar ratio of 1:1, and the mixing reaction was stirred. After rising to room temperature, the stirring reaction continued for 12 h. After the product was separated through the column, the obtained product and 1, 3-dichloropropane were added into the pressure bottle according to 2:1, and isopropanol was added as the solvent. After the stirring reaction at 120° C. for 12 h, the solvent was removed by vacuum distillation, and the resulting product was washed with acetone for three times and freeze-dried to obtain the sulfur-free Gemini quaternary ammonium salt. The sulfur-free Gemini quaternary ammonium salt was C10 quaternary ammonium salt without sulfur, denoted by code C10-Cl. The structural formula of C10-Cl was shown as follows:

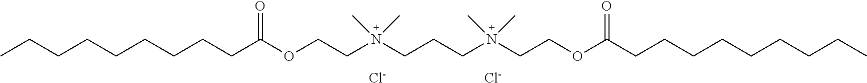

(2) According to the molar ratio of 1:3, the above C10-Cl Gemini quaternary ammonium salt and AgNO₃ were added to water, prepared into an aqueous solution with a concentration of 1.0 wt %, stirred for 12 h, and filtered to obtain the supernatant, that was, the uncomplexed silver ion Gemini quaternary ammonium salt was obtained, denoted by the code C10-NO₃—Ag⁺.

In order to verify whether there is a complex reaction between Ag⁺ and the C10-Cl, the C10-Cl and AgNO₃ were added to the water according to the molar ratio of 1:0, 1:1, 1:2 and 1:4 respectively, and the operation was performed according to step (2). According to the molar ratio 1:0, 1:1, 1:2, 1:3 and 1:4, the uncomplexed silver ion Gemini quaternary ammonium salts were obtained by the reaction between C10-Cl and AgNO₃.

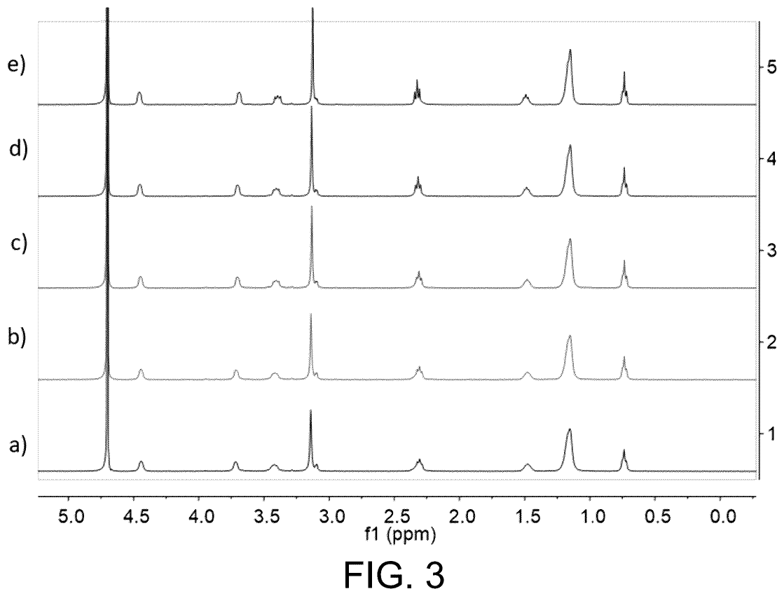

The uncomplexed silver ion Gemini quaternary ammonium salts were tested by nuclear magnetic hydrogen spectrum (FIG. 3). The silver ion uncomplexes Gemini quaternary ammonium salts obtained from the reaction of C10-Cl with molar ratio of 1:0 and 1:3 with AgNO₃ were C10-Cl and C10-NO₃—Ag⁺.

FIG. 3 showed the nuclear magnetic hydrogen spectra results of the silver ion uncomplexes Gemini quaternary ammonium salt obtained by the reaction of C10-Cl with different molar ratio with AgNO₃, where, a) was the 1H NMR spectra of the silver ion uncomplex Gemini quaternary ammonium salt obtained by the reaction of C10-Cl and AgNO₃ with the molar ratio of 1:0, which is actually the 1H NMR spectra of C10-Cl; b) was the 1H NMR spectra of the silver ion uncomplex Gemini quaternary ammonium salt obtained by the reaction of C10-Cl and AgNO₃ with the molar ratio of 1:1; c) was the 1H NMR spectra of the silver ion uncomplex Gemini quaternary ammonium salt obtained by the reaction of C10-Cl and AgNO₃ with the molar ratio of 1:2; d) was the 1H NMR spectra of the silver ion uncomplexes Gemini quaternary ammonium salt obtained by the reaction of C10-Cl and AgNO₃ with the molar ratio of 1:3, which is actually the 1H NMR spectra of C10-NO₃—Ag⁺; e) was 1H NMR spectra of the silver ion uncomplexes Gemini quaternary ammonium salt obtained by the reaction of C10-Cl and AgNO₃ with the molar ratio of 1:4.

From FIG. 3, it could be observed that when C10-Cl reacted with AgNO₃, the 1H NMR spectra did not change with adding more AgNO₃, indicating that there was no complex reaction between Ag⁺ and C10-Cl.

### Performance Test:

1. Bactericidal Performance of Gemini Quaternary Ammonium Salts with Different Lengths of Carbon Chains:

Referring to 2.1.1.7.4 suspension quantitative bactericidal test (2002 edition of Disinfection Technical Specifications), neutralizer was prepared: 2.1% lecithin+2.0% glucose+0.5% sodium thiosulfate+1.5% tween-80+0.5% peptone+1.2% sodium sulfite aqueous solution. C6-S—Cl (Example 1), C8-S—Cl (Example 3) and C10-S—Cl (Example 5) were diluted into 0.0015625% and 0.00078125% aqueous solutions, and the bactericidal experiment was carried out for 30 min to Escherichia coli. The results were shown in Table 1.

TABLE 1
Bactericidal properties of Gemini quaternary ammonium
salts with different length carbon chains

| Sample | Concentration | Treated (cfu/mL) | Sterilizing rate |
|---|---|---|---|
| C6-S-Cl | 0.0015625% | 3.2 × 10³ | 99.989% |
| | 0.00078125% | 4.0 × 10⁵ | 98.621% |
| C8-S-Cl | 0.0015625% | 90 | >99.999% |
| | 0.00078125% | 1.9 × 10² | >99.99% |
| C10-S-Cl | 0.0015625% | 5.8 × 10² | >99.99% |
| | 0.00078125% | 4.6 × 10⁴ | 99.841% |

From Table 1, it could be found that the prepared Gemini quaternary ammonium salts with different chain lengths all had certain killing effects on Escherichia coli. Especially, C8-S—Cl has a bactericidal rate above 99.99%, showing the best bactericidal performance.

2. Bactericidal Performance of Gemini Quaternary Ammonium Salts with Different Anionic:

Referring to 2.1.1.7.4 suspension quantitative bactericidal test (2002 edition of Disinfection Technical Specifications), neutralizer was prepared: 2.1% lecithin+2.0% glucose+0.5% sodium thiosulfate+1.5% tween-80+0.5% peptone+1.2% sodium sulfite aqueous solution. C8-S—Cl (Example 3), C8-S—Br (Example 4) and C8-S—NO₃ (Example 7) were diluted into 0.0015625% and 0.00078125% aqueous solutions, and the bactericidal experiment was carried out for 30 min to Escherichia coli. The results were shown in Table 2.

TABLE 2
Bactericidal properties of different
anionic quaternary ammonium salts

| Sample | Concentration | Treated (cfu/mL) | sterilizing rate |
|---|---|---|---|
| C8-S-Cl | 0.0015625% | 79 | >99.999% |
| | 0.00078125% | 1.7 × 10³ | >99.99% |
| C8-S-Br | 0.0015625% | 75 | >99.999% |
| | 0.00078125% | 1.1 × 10³ | >99.99% |
| C8-S-NO₃ | 0.0015625% | 13 | >99.999% |
| | 0.00078125% | 1.4 × 10² | >99.999% |

From Table 2, It could be found that different anionic quaternary ammonium salts had high killing effect on Escherichia coli. Especially, nitric quaternary ammonium salt had a bactericidal rate above 99.999%, showing the best bactericidal performance.
3. Bactericidal Performance of Gemini Quaternary Ammonium Salt Complexed with Different Metal Ions:

Referring to 2.1.1.7.4 suspension quantitative bactericidal test (2002 edition of Disinfection Technical Specifications), neutralizer was prepared: 2.1% lecithin+2.0% glucose+0.5% sodium thiosulfate+1.5% tween-80+0.5% peptone+1.2% sodium sulfite aqueous solution. C8-S—Cl (Example 3), C8-S—NO₃—Ag⁺ (Example 3), C8-S—CI—Cu²⁺ (ratio 1), C8-S—CI—Mn²⁺ (ratio 2), C8-S—CI—Fe³⁺ (ratio 3), AgNO₃, CuCl₂, MnCl₂, and FeCl₃ were dissolved to 0.0015625% and 0.00078125% aqueous solution. The bactericidal experiment was carried out for 30 min to Escherichia coli, and the results were shown in Table 3.

TABLE 3
Bactericidal properties of Gemini quaternary ammonium
salts complexed with different metal ions

| | | Treated | |
|---|---|---|---|
| Sample | Concentration | (cfu/mL) | sterilizing rate |
| C8-S-Cl | 0.0015625% | 86 | >99.999% |
| | 0.00078125% | 1.8 × 10³ | >99.99% |
| C8-S-CI-Cu²⁺ | 0.0015625% | 1.02 × 10² | >99.999% |
| | 0.00078125% | 1.6 × 10³ | >99.99% |
| C8-S-CI-Mn²⁺ | 0.0015625% | 96 | >99.999% |
| | 0.00078125% | 1.8 × 10³ | >99.99% |
| C8-S-CI-Fe³⁺ | 0.0015625% | 93 | >99.999% |
| | 0.00078125% | 1.7 × 10³ | >99.99% |
| C8-S-NO₃-Ag⁺ | 0.0015625% | 13 | >99.999% |
| | 0.00078125% | 79 | >99.999% |
| CuCl₂ | 0.0015625% | 3.2 × 10⁷ | No bactericidal effect |
| | 0.00078125% | 4.9 × 10⁸ | No bactericidal effect |
| MnCl₂ | 0.0015625% | 2.3 × 10⁷ | No bactericidal effect |
| | 0.00078125% | 8.9 × 10⁷ | No bactericidal effect |
| FeCl₃ | 0.0015625% | 4.3 × 10⁷ | No bactericidal effect |
| | 0.00078125% | 6.2 × 10⁸ | No bactericidal effect |
| AgNO₃ | 0.0015625% | 2.2 × 10⁷ | No bactericidal effect |
| | 0.000781250% | 1.3 × 10⁸ | No bactericidal effect |

From Table 3, it could be found that the introduction of Cu²⁺, Mn²⁺ and Fe³⁺ to C8-S—CI has no significant effect on the bactericidal performance. CuCl₂, MnCl₂, FeCl₃ and AgNO₃ solutions did not show any antibacterial performance at this concentration. It was interesting to find that the Gemini quaternary ammonium salt with Ag⁺ complex could significantly improve the bactericidal performance.

4. Bactericidal Performance of Gemini Quaternary Ammonium Salt with and without Ag⁺ Complex:

Referring to 2.1.1.7.4 suspension quantitative bactericidal test (2002 edition of Disinfection Technical Specifications), neutralizer was prepared: 2.1% lecithin+2.0% glucose+0.5% sodium thiosulfate+1.5% tween-80+0.5% peptone+1.2% sodium sulfite aqueous solution. C8-S—Cl (Example 3), C8-S—NO₃ (Example 7) and C8-S—NO₃—Ag⁺ (Example 3) were configured into 0.0015625%, 0.00078125% and 0.000390625% aqueous solutions. The bactericidal experiment was carried out for 30 min to Escherichia coli, and the results were shown in Table 4.

TABLE 4
Bactericidal properties of Gemini quaternary
ammonium salt with and without Ag⁺ complex

| | | | sterilizing |
|---|---|---|---|
| Sample | Concentration | Treated (cfu/mL) | rate |
| C8-S-NO₃-Ag⁺ | 0.0015625% | 9 | >99.999% |
| | 0.00078125% | 62 | >99.999% |
| | 0.000390625% | 2.5 × 10³ | >99.99% |
| C8-S-NO₃ | 0.0015625% | 13 | >99.999% |
| | 0.00078125% | 2.5 × 10² | >99.99% |
| | 0.000390625% | 4.5 × 10⁴ | >99.99% |

From Table 4, it could be found that the bactericidal performance of the quaternary ammonium salt was significantly improved with Ag⁺ complex.

### 5. Bactericidal Performance Test of Gemini Quaternary Ammonium Salts of Complex and Uncomplex Silver Ions:

Referring to 2.1.1.7.4 suspension quantitative bactericidal test (2002 edition of Disinfection Technical Specifications), neutralizer was prepared: 2.1% lecithin+2.0% glucose+0.5% sodium thiosulfate+1.5% tween-80+0.5% peptone+1.2% sodium sulfite aqueous solution. The bactericidal experiment was carried out on E. coli for 30 min by preparing the silver-ion complex Gemini quaternary ammonium salt (Example 3), C10 chlorinated sulfur-free Gemini quaternary ammonium salt (Comparative example 4) and uncomplex silver-ion Gemini quaternary ammonium salt (Comparative example 4) into 0.0015625%, 0.00078125% and 0.000390625% aqueous solutions. The bactericidal rate results were shown in Table 5.

TABLE 5
Bactericidal properties of Gemini quaternary ammonium
salts of complex and uncomplex silver ions

| Sample | Concentration | Treated (cfu/mL) | sterilizing rate |
|---|---|---|---|
| C10-Cl | 0.0015625% | 1.2 × 10² | >99.999% |
| | 0.00078125% | 3.5 × 10³ | >99.99% |
| | 0.000390625% | 4.3 × 10⁵ | 99.978% |
| C8-S-Cl | 0.0015625% | 82 | >99.999% |
| | 0.00078125% | 1.8 × 10³ | >99.99% |
| | 0.000390625% | 2.6 × 10⁵ | 99.985% |
| C10-NO₃-Ag⁺ | 0.0015625% | 53 | >99.999% |
| | 0.00078125% | 1.8 × 10² | >99.99% |
| | 0.000390625% | 1.6 × 10⁵ | 99.986% |
| C8-S-NO₃-Ag⁺ | 0.0015625% | 12 | >99.999% |
| | 0.00078125% | 1.03 × 10² | >99.999% |
| | 0.000390625% | 2.5 × 10³ | >99.99% |

Table 5 is a comparison of the bactericidal properties of the mixed solution of The Gemini quaternary ammonium salts containing sulfur atoms and those without sulfur atoms and their silver ions (mixed equivalent of 1:3). From Table 5, it could be found that the sulfur had no significant effect on the bactericidal performance of the Gemini quaternary ammonium salt, but there was a significant difference when it was mixed with the aqueous solution of silver ions. The bactericidal performance of the Gemini quaternary ammonium salt containing sulfur atoms had been improved to a certain extent, thanks to the presence of sulfur atoms, silver ions and the Gemini quaternary ammonium salt had been fully complexed. The bactericidal properties of the quaternary ammonium salt after complexation were improved.

The described embodiments were only preferred embodiments of the present invention/disclosure, and it should be noted that various alterations and improvements may be made therein by those of ordinary skill in the art without departing from the principle of the present invention/disclosure and should also fall within the scope of protection of the present invention/disclosure.

1. A silver ion complex Gemini quaternary ammonium salt, comprising structural formula: 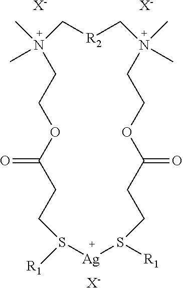
R₁, which is a saturated straight-chain alkyl group having 6-12 carbon atoms; R₂, which is an alkyl group having 1-8 carbon atoms; X, which is an anionic group.

2. The silver ion complex Gemini quaternary ammonium salt of claim 1, wherein R₁ is a saturated straight-chain alkyl group having 8 carbon atoms.

3. The silver ion complex Gemini quaternary ammonium salt of claim 1, wherein R₂ is a methylene group.

4. The silver ion complex Gemini quaternary ammonium salt of claim 1, wherein X is selected from the group consisting of NO₃, OAc, SCN, BF₄, OTf, HSO₄, PF₆, BPh₄, Cl, Br, and I.

5. A method for preparing a silver ion complex Gemini quaternary ammonium salt, wherein the method comprises:
S1: performing a Michael addition reaction and a nucleophilic substitution reaction on dimethylaminoethyl acrylate, a long-chain thiol, and a dihaloalkane to obtain the Gemini quaternary ammonium salt;
S2: reacting the obtained Gemini quaternary ammonium salt with a silver salt to form the silver ion complexed Gemini quaternary ammonium salt.

6. The method of claim 5, wherein in step S1 the long-chain thiol has the following structural formula: 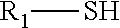
wherein R₁ is a saturated straight-chain alkyl group having 6-12 carbon atoms; and
the dihaloalkane has the following structural formula: 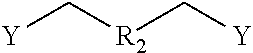
wherein R₂ is alkyl group having 1-8 carbon atoms, and Y is Cl, Br or I.

7. The method of claim 5, wherein the long-chain thiol is 1-Hexanethiol, 1-Heptanthiol, 1-Octanethiol, 1-Nononythiol, or 1-Decanethiol.

8. The method of claim 5, wherein the dihaloalkane is 1, 3-dichloropropane or 1, 3-dibromopropane.

9. The method of claim 5, wherein the reaction temperature is between 50-150° C. and the reaction time is between 6-18 hours in step S1.

10. The method of claim 5, wherein the silver salt used is silver nitrate in step S2.

11. The method of claim 10, wherein the molar ratio of silver salt and the Gemini quaternary ammonium salt is at least 3:1.

12. The method of claim 5, wherein the reaction temperature is 10-30° C. and the reaction time is 6-18 hours in step S2.

13. A use of the silver ion complex Gemini quaternary ammonium salt of claim 1 in a disinfectant.

14. A use of the silver ion complex Gemini quaternary ammonium salt prepared according to the method of claim 5 in a disinfectant.
